(19) United States
(12) Patent Application Publication
(10) Pub. No.: US 2010/0216145 A1
(43) Pub. Date: Aug. 26, 2010

(54) Assay for Detecting Circulating Free Nucleic Acids

(75) Inventor: Amos Duvdevani, Beer Sheva (IL)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Mor Research Application Ltd., Tel Aviv (IL)

(21) Appl. No.: 12/667,186

(22) PCT Filed: Dec. 4, 2008
(86) PCT No.: PCT/IL2008/001584

§ 371 Date: May 6, 2010

Related U.S. Application Data
(60) Provisional application No. 60/996,900, filed on Dec. 10, 2007.

(51) Int. Cl. C12Q 1/68

(52) U.S. Cl. 435/6

(57) ABSTRACT

This invention is directed, inter alia, to methods and kits for rapid, easy and cost-effective methods of all free nucleic acid quantification in inter alia, biological fluid samples.

### BACKGROUND OF THE INVENTION

There is an increase in the amount of free DNA in the blood that is correlated with cell death, as a function of tissue injury or inflammatory responses. An increase in free DNA in the blood as a result of many diseases has been seen, including autoimmune disease, stroke, cancer and cardiovascular disease. Free DNA levels have been considered to be a telling prognostic for these and other diseases, yet the methodology to quantitatively assess free circulating DNA levels is expensive and time consuming.

There is an increase in the amount of free DNA in the blood that is correlated with cell death, as a function of tissue injury or inflammatory responses, or other diseases. For example, one characteristic property of cancer and other cell proliferative diseases is an increased amount of free floating, circulating DNA in blood and/or serum. Cell death caused by for example toxic doses of bacterial lipopolysaccharide, and toxic chemicals triggers the release of products of chromatin catabolism, particularly of DNA into extracellular spaces, which may also be detected by quantification of serum or blood DNA levels. An increase in free DNA in the blood as a result of many diseases has been seen, including autoimmune diseases, stroke, cancer and cardiovascular disease.

Free DNA levels have been considered to be a telling prognostic for these and other diseases, yet the methodology to quantitatively assess free circulating DNA levels is expensive and time consuming.

### SUMMARY OF THE INVENTION

In one embodiment, this invention provides a method of quantifying the nucleic acid concentration in a biological fluid of a subject, the method comprising the steps of:
a) mixing a biological fluid sample with a detectable nucleic acid intercalating agent, wherein said mixing is conducted in the absence of prior nucleic acid extraction;
b) detecting said moiety; and
c) correlating detection of said moiety with a value reflective of the concentration of nucleic acid in said biological fluid sample;

In some embodiments, the method employs serially diluted, bodily fluid samples. In some embodiments, the detectable nucleic acid intercalating agent comprises a detectable moiety or in some embodiments, the detectable nucleic acid intercalating agent is fluorescent, and in one embodiment, detecting is conducted with the use of a fluorometer. In one embodiment, the detectable nucleic acid intercalating agent comprises SYBR Gold© or SYBR Green©.

In one embodiment, the nucleic acid is DNA.

In one embodiment, the method is conducted in parallel to mixing a second biological fluid sample obtained from a second subject, and said correlating results in said value representing a standard for said method. In one embodiment, the method further comprises mixing a second fluid sample comprising a known concentration of nucleic acid with said detectable agent and correlating detection with a value equal to said known concentration. In one embodiment, correlating includes assigning a value to said biological fluid sample based on the comparative detection with that obtained for said second fluid sample.

In one embodiment, the biological fluid is a bodily fluid. In another embodiment the biological fluid is a cell lysate or organ homogenate. In some embodiments, the biological fluid is a lavage.

In one embodiment, the subject has or is predisposed to a disease or disorder. In one embodiment, the method further comprises diagnosing the presence of said disease or disorder based on said value obtained. In one embodiment, the method further comprises predicting the severity of said disease or disorder based on said value obtained. In one embodiment, the method further comprises assessing response of a subject to treatment of said disease or disorder, based on said value obtained. In one embodiment, the disease or disorder comprises a tissue injury, an infection, an inflammatory response, neoplasia or preneoplasia. In one embodiment, tissue injury comprises myocardial infarction.

In one embodiment, this invention provides a method of quantifying the in vitro nucleic acid concentration in a tissue culture fluid, the method comprising the steps of:
a) mixing a tissue culture fluid sample with a detectable nucleic acid intercalating agent, wherein said mixing is conducted in the absence of prior nucleic acid extraction;
b) detecting said moiety; and
c) correlating detection of said moiety with a value reflective of the

In some embodiments, the method is conducted in parallel to mixing a second tissue culture fluid sample obtained from a source subjected to an alternative culture condition than that of said first tissue culture fluid sample.

In one embodiment, the invention provides a method of quantifying the residual nucleic acid concentration in a recombinant protein bioreactor fluid, the method comprising the steps of:
a) mixing a fluid sample obtained from a bioreactor for the preparation of recombinant proteins with a detectable nucleic acid intercalating agent, wherein said mixing is conducted in the absence of prior nucleic acid extraction;
b) detecting said moiety; and
c) correlating detection of said moiety with a value reflective of the concentration of nucleic acid in said fluid sample.

In some embodiments, the method indicates bioreactor efficiency.

In one embodiment, this invention provides a kit for the quantification of the nucleic acid concentration of a bodily fluid of a subject, said kit comprising:
a) a detectable nucleic acid intercalating agent;
b) a diluent; and
c) a series of solutions comprising nucleic acid samples in said diluent, wherein the concentration of each of the nucleic acid samples in said series is known;
   whereby a bodily fluid sample is mixed with said detectable nucleic acid intercalating agent in parallel to mixing said agent with said series, and detection of said agent in said series serves as a standard for arriving at a value reflective of the concentration of nucleic acid in said bodily fluid sample.

In one embodiment, the detectable nucleic acid intercalating agent comprises a detectable moiety. In one embodiment, the detectable nucleic acid intercalating agent is fluorescent. In one embodiment, the kit optionally comprises a container suitable for accommodating said series of solutions and said bodily fluid sample and wherein said container may by applied to a fluorometer. In one embodiment, the detectable nucleic acid intercalating agent comprises SYBR Gold© or SYBR Green©. In one embodiment, the nucleic acid samples comprise DNA. In one embodiment, the kit comprises a container suitable for the assay of urine, blood or a component thereof, lavage fluid or a combination thereof.

All publications, patents, and patent applications mentioned herein are hereby incorporated by reference in their entirety as if each individual publication or patent was specifically and individually indicated to be incorporated by reference. In case of a conflict between the specification and an incorporated reference, the specification shall control. Where number ranges are given in this document, endpoints are included within the range. Furthermore, it is to be understood that unless otherwise indicated or otherwise evident from the context and understanding of one of ordinary skill in the art, values that are expressed as ranges can assume any specific value or subrange within the stated ranges, optionally including or excluding either or both endpoints, in different embodiments of the invention, to the tenth of the unit of the lower limit of the range, unless the context clearly dictates otherwise. Where a percentage is recited in reference to a value that intrinsically has units that are whole numbers, any resulting fraction may be rounded to the nearest whole number.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 describes fluorescence as a function of DNA concentration, in serum samples probed with SYBR Gold. Concentrations of as little as 50 ng/ml of DNA were detected. Commercial Salmon sperm DNA was dissolved at various concentrations in four different fluids: A. 20% solution of DNase-treated pooled serum from 10 healthy donors in PBS. B. 2% solution of bovine serum albumin (BSA) in PBS. C. Fresh heparinized whole blood from a healthy donor and D. Pooled urine from 10 healthy donors. Urine was buffered to pH 7.4 with 10 mM HEPES. DNA solutions were added in duplicates to black 96 well plates, SYBR® Gold was added to each well (1:10000) and fluorescence was measured at 535 nM (F535) by a plate reader fluorometer.

FIG. 2A and FIG. 2B are side-by-side comparisons of fluorescence of SYBR Gold and SYBR Green mixed with serially diluted salmon DNA in 20% normal pooled human sera. FIG. 2C demonstrates DNA detection in whole blood with SYBR gold. FIG. 2D shows linear fluorescence intensity as a function of DNA concentration in the presence of the florescent dye EvaGreen®. For this experiments DNA was diluted in phosphate buffered saline (PBS) containing 2% bovine serum albumin (BSA) and EvaGreen® was added in final dilution of 1:1000.

FIG. 3A describes fluorescence as a function of DNA concentration, in peritoneal lavage fluid collected from mice challenged intra-peritoneally with E. coli. FIG. 3B demonstrates that DNA concentrations correlated well with the levels of IL-6 or TNF ( FIG. C) markers that reflect the intensity of a destructive inflammatory process in lavage fluid and in serum.

FIG. 4A describes serum troponin correlation with DNA detection. FIG. 4B demonstrates that treatment of the serum sample with DNase abolished fluorescence. Random serum samples (0.5 ml) were treated with DNase (500 U) or RNase (100 U) (FIGS. 4C and 4D). C. Fluorescence of one representative serum. D. Fluorescence of sera after incubation with RNase (=5) or DNase (n=9) in relation to the corresponding sera not incubated with nuclease. *** indicates p<0.001. FIGS. 4E-G show DNA quantification of samples from hospitalized patients with acute myocardial infarction (MI) at different hours following their arrival at the emergency room of the hospital. FIG. 4H-J depict the DNA level (H), distribution, (I) and patient outcome (J) of 200 subjects who were evaluated in this setting, as compared to healthy volunteers.

FIG. 5 describes a side-by-side DNA quantification of samples in which DNA was subjected to a prior extraction step, or not. Panels A and B quanitifies DNA isolated from whole blood of a normal healthy donor and extracted, per the QIAamp DNA blood Kit (Qiagen) and quantified by SYBR Gold assay (5A) or QPCR of the β-globin gene. Panel C describes DNA quantification by SYBR Gold assay in serum, where the DNA was not subjected to a prior extraction step. Panel D describes the linear correlation between the SYBR® Gold assay and β-globin QPCR assay. Human DNA was purified from leukocytes of a healthy volunteer and quantified by optical density (260 nM) using a nanodrop spectrophotometer. Serial dilution of DNA concentration was then determined by the SYBR® Gold assay (F535) and by real time PCR (QPCR) using specific primers for β-globin.

FIG. 6 describes the effect of serum concentration on F535 background and quenching. Pooled human serum was preincubated with DNase and diluted with PBS to various concentrations; same amount of salmon sperm DNA was added to all solutions resulting in a final concentration of 1140 ng/ml. Serum solutions at same concentrations not containing DNA were used to determine background fluorescence. A. Total and background fluorescence of serum solutions B. Calculated % quenching of the specific DNA signal [100−100×(Total F535−Background F535)/total F535]. Assay was performed in triplicates, ** indicates p<0.01 comparing serum solution to PBS without serum.

FIG. 7 demonstrates the effect of storage conditions on the assay. A. Blood from 7 healthy volunteers was collected into commercial gel tubes (8 tubes per donor). From each donor 5 tubes were stored at room temperature (RT) and 3 tubes at 4° C. Tubes were centrifuged and sera were collected for the DNA assay at indicated time points. B. Aliquots of 10 different sera (3 low, 4 elevated and 3 high DNA concentrations) were and incubated for 24 hrs at RT or frozen and thawed 5 times and then assayed for DNA. Assays were performed in triplicates. Readings of different time points were compared with readings at time zero. *** indicates p<0.001.

FIG. 8 shows intra-day and intra-assay variation: To assess the variation of the assay, three sera were used containing normal, elevated and high DNA concentrations (197, 1096 and 4107 ng/ml, respectively) A. Intra-day variation of the assay was assessed by comparing readings of 12 assays of each sample in duplicates done independently on separate plates at different times over one working day. B. Day to day variation was assessed by comparing readings of 12 aliquots of each sample. Aliquots have been frozen and assayed on different days. For this assay, serum of three donors was used with low, elevated and high DNA concentrations (383, 1152 and 2735 ng/ml, respectively). Median value of the assays is indicated by the line inside the box. The Box indicates the distribution of 50% of the results and the bar above and below the box indicates 25% of the data. C Normal reference values were obtained by analysis of sera from 47 healthy volunteers. The volunteers were mostly students which declared to be healthy and with no chronic disease. The cohort consisted of 22 women and 25 man with an average age of 26.3±4.7 years. D. presents statistical analysis.

FIG. 9A demonstrates DNA quanitification in subjects with viral infection, where DNA levels detected are higher in active EBV and CMV infection, as opposed to controls. FIG. 9B demonstrates that DNA quantification correlated well with viral load in HIV infected patients. FIG. 9C demonstrates quantification in subjects with sepsis. FIG. 9D demonstrates quantification in subjects with active peritonitis, and the correlation between leukocyte number in peritoneal fluid and DNA concentration.

FIG. 10 demonstrates quantification in a subject recovering from acute graft rejection following kidney transplantation, with DNA levels correlating well with creatinine levels.

FIG. 11 demonstrates quantification of circulating DNA levels in trauma patients and its correlation with clinical complications arising in particular subjects.

FIG. 12 shows DNA quantification in cancer subjects and cancer models. A. CFD levels were elevated in patients with colon cancer, one week before tumor removal. B. Elevated circulating DNA levels correlate with tumor size in mice inoculated intra footpad with an MCA-2 fibrosarcoma cell line with 1.0×10⁶ cells/mouse (N=10).

FIG. 13 shows assay efficacy on cell lysates. Cultured fibroblast cells (L-cells) seeded at various numbers in triplicates (0, 40, 60, 80, 100, 120, 150 and 200×103 cells/well) in 24 well plates with 1 ml of DMEM medium containing 10% fetal calf serum. Cell lysis was induced with a detergent (0.1% NP40) and gentle agitation for 30 minutes. Supernatants were collected and assayed for free DNA and LDH activity. A. Supernatant free DNA (F535). B. Supernatant LDH activity. C. Correlation between supernatant free DNA and LDH activity.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

This invention is directed, inter alia, to methods and kits for rapid, easy and cost-effective methods of nucleic acid quantification in bodily fluid samples, tissue culture fluid samples and bioreactor fluid samples.

Healthy human beings have free DNA in bodily fluids however such DNA is at a low level, reportedly in the range of 2-30 ng/ml. Diseased human beings, for example, cancer patients, have been found to exhibit increased levels of free DNA in bodily fluids.

In one embodiment, this invention provides a method of quantifying the nucleic acid concentration in a biological fluid of a subject, the method comprising the steps of:
a) mixing a biological fluid sample with a detectable nucleic acid intercalating agent, wherein said mixing is conducted in the absence of prior nucleic acid extraction;
b) detecting said moiety; and
c) correlating detection of said moiety with a value reflective of the concentration of nucleic acid in said biological fluid sample;

In some embodiments, the invention is concerned with the measurement of nucleic acids in biological fluids. In some embodiments, the term “nucleic acid” refers to a covalently linked sequence of nucleotides (i.e., ribonucleotides for RNA and deoxyribonucleotides for DNA) in which the 3′ position of the pentose of one nucleotide is joined by a phosphodiester group to the 5′ position of the pentose of the next. The term “nucleic acid” includes, without limitation, single- and double-stranded polynucleotide. The term “nucleic acid” as it is employed herein embraces all forms of nucleic acids, e.g., DNA, RNA, PNA, combinations of these, etc.

In some embodiments, the methods detect and quantify free DNA in biological fluids. In some embodiments, the term ‘free DNA’ refers to extracellular deoxynucleic acids, for example unbound DNA or circulating nucleic acids as present in bodily fluids as defined above. The DNA can, nevertheless, be bound to proteins in the bodily fluid, this will also be understood to represent embodiments of “free DNA” in the context of the present invention. In some embodiments, the DNA free in the bodily fluid is derived from single cells or clumps of cells that are derived from organs or tissues (e.g. lung cells that are expectorated) and have lysed, releasing their DNA. The DNA that is released from these cells in said bodily fluid will also be understood as “free DNA” in the context of the present invention.

The invention provides methods and/or kits for quantifying free DNA in bodily fluids. In some embodiments, the term “ biological fluid” refers to a liquid taken from a biological source and includes, for example, blood, serum, plasma, sputum, lavage fluid, cerebrospinal fluid, urine, semen, sweat, tears, saliva, or others.

In some embodiments, the bodily fluid refers to whole blood, blood plasma, blood serum, urine, sputum, ejaculate, semen, tears, sweat, saliva, lymph fluid, bronchial lavage, leukophoresis samples, pleural effusion, peritoneal fluid, meningal fluid, amniotic fluid, glandular fluid, fine needle aspirates, nipple aspirate fluid, spinal fluid, conjunctival fluid, vaginal fluid, duodenal juice, pancreatic juice, bile, cerebrospinal fluid or mucus secretions from a desired subject. In some embodiments, the term “fluid” for sample in the methods and via the kits of this invention, refers to a tissue homogenate, cell culture or bioreactor fluid sample, as described further hereinbelow.

In some embodiments, the biological fluids are from mammalian subjects, where a sample may be obtained from differing sources, including, but not limited to, samples from different individuals, different developmental stages of the same or different individuals, different diseased individuals (e.g., individuals with cancer or suspected of having a genetic disorder), normal individuals, different disease stages of the same or different individuals, individuals subjected to different disease treatment, individuals subjected to different environmental factors, or individuals with predisposition to a pathology, or individuals with exposure to an infectious disease agent (e.g., HIV).

For clinical identification of pathogens, it is desirable to recover the DNA/RNA from bodily fluids, tissues or excretions containing the bacteria/virus. Such samples may be derived, for example, from feces, urine, blood, sputum, wound exudates, or other sources.

In one embodiment, the sample is collected from a pregnant female, for example a pregnant woman. According to this aspect, and in one embodiment, the sample can be analyzed using the methods described herein to prenatally diagnose chromosomal abnormalities in the fetus. The sample can be collected from biological fluids, for example the blood, serum, villus sampling, or some fraction thereof.

In some embodiments, reference to the terms “blood,” “plasma” and “serum” are to be taken to expressly encompass fractions or processed portions thereof. Similarly, where a sample is taken from a biopsy, swab, smear, etc., the “sample” expressly encompasses a processed fraction or portion derived from the biopsy, swab, smear, etc.

In some embodiments, the bodily fluid is obtained from a single subject or individual, or in some embodiments, from pooled subjects. In some embodiments, the term “individual” or “subject” refers to a human subject as well as a non-human subject such as a mammal, an invertebrate, a vertebrate, a rat, a horse, a dog, a cat, a cow, a chicken, a bird, a mouse, a rodent, a primate, a fish, a frog, a deer. In some embodiments, the subject may be infected, with for example, a fungus, a yeast, a parasite, a bacteria, or a virus. The examples herein are not meant to limit the methodology of the present invention to a human subject only, as the instant methodology is also useful in the fields of veterinary medicine, animal sciences, research laboratories and such.

In some embodiments, the method comprises mixing a bodily fluid sample with a detectable nucleic acid intercalating agent.

In some embodiments, the term “mixing” refers to contact proximity, for example, dispensing of a fluid detectable nucleic acid intercalating agent in a container containing a sample of the bodily fluid, or vice versa. In some embodiments, mixing may comprise more extensive agitation of the fluid, with any aid, such as, for example, conventional mixers, the use of stirring aids, the use of vortex machinery, sonication, or any means known in the art. No means of mixing is to be considered precluded, nor is any limitation imposed upon the time or amount of mixing necessary for the creation of proximity between the fluid sample and the intercalating agent, such that the intercalating agent may intercalate within nucleic acids present in the bodily fluid sample.

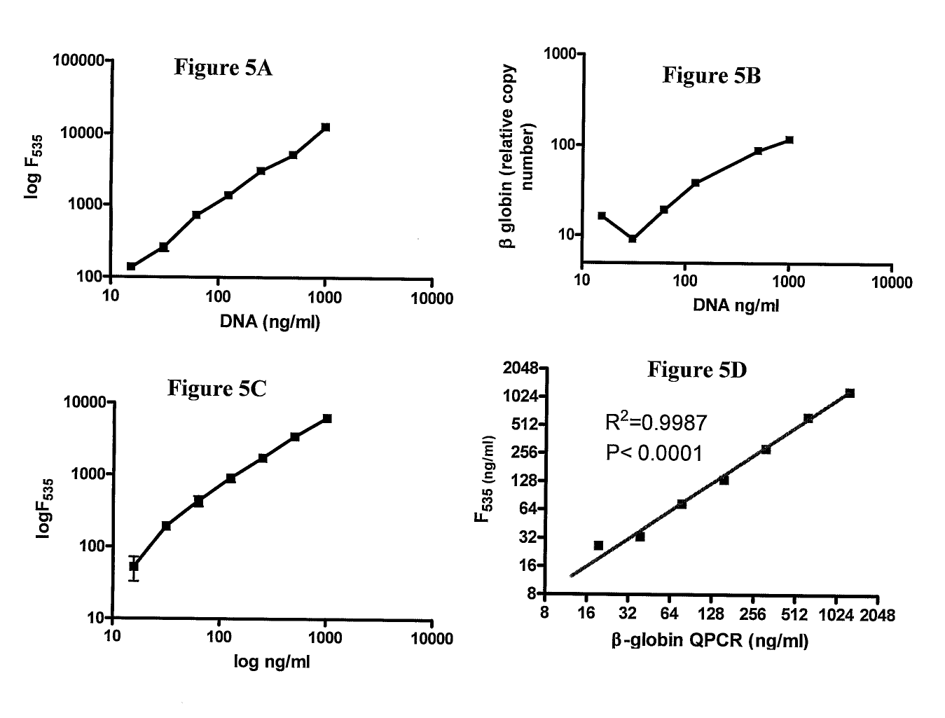

Surprisingly, in the present invention, it was found that contacting bodily fluid samples comprising nucleic acids, with a nucleic acid intercalating agent, without prior nucleic acid extraction was as sensitive, and in some embodiments, more sensitive, in quantifying the nucleic acid concentration in the sample, than samples whose nucleic acids had been previously subjected to extraction (FIG. 5). The lack of necessity for such an extraction step increases efficiency and ease of the quantification assay, and reduces cost, such that there is a clear advantage to such assays.

The methods and kits of this invention make use of a nucleic acid intercalating agent. In some embodiments, the term “intercalating” or grammatical forms thereof refers to the insertion of a compound between adjacent base pairs of a strand of DNA. For example, and in some embodiments, the term “intercalating” refers to the insertion of planar aromatic or heteroaromatic compounds between adjacent base pairs of double stranded DNA (dsDNA).

In some embodiments, the intercalating agent is one in which a change in fluorescence occurs upon binding to a nucleic acid. In some embodiments, the intercalating agent is one which fluoresces upon binding to DNA, or in some embodiments exhibits a marked increase in fluorescence upon DNA binding.

In some embodiments, the intercalating agent is a phenanthridium compound, as described in U.S. Pat. Nos. 5,436,134, 5,582,984, 5,808,077, 5,658,751, 6,664,047, fully incorporated herein in their entirety. In some embodiments, the intercalating agent is a cyanine compound, for example a dimeric cyanine stain. In some embodiments, the cyanine compound is a SYBR® stain, Picogreen®, Oligreen® or Ribogreen® or a POPO®, BOBO®, YOYO®, TOTO®, JOJO® or LOLO® stains or TO-PRO® stains (Molecular Probes/Invitrogen Inc.) or EvaGreen®. In some embodiments, the intercalating agent is ethidium bromide, propidium iodide, Quinolinium, 1-1′-[1,3-propanediylbis [(dimethyliminio)-3,1-propanediyl]] bis[4-[(3-methyl-2(3H)-benzothiazolylidene)methyl]]-, tetraiodide}, or 4-[(3-methyl-2 (3H)-benzoxazolylidene)methyl]-1-[3-(trimethylammonio)propyl]-, diiodide}.

The intercalating agents used in the methods/kits of this invention comprise a detectable moiety. In some embodiments, the phrase “comprise a detectable moiety” refers to the association of the moiety with the intercalating agent, for example by covalent or non-covalent bonds. In some embodiments, the phrase “comprise a detectable moiety” refers to the agent itself being detectable, for example, the agent itself fluoresces upon DNA binding.

The methods comprise detecting the moiety by any means known in the art, suitable for detection of the particular moiety. For example, and in some embodiments, the detectable moiety is fluorescent upon binding, and detection is accomplished with the aid of a fluorimeter. In some embodiments, spectrophotometric detection may be used for detectable changes in absorbance upon interaction of the detectable moiety with the nucleic acid. In some embodiments, detection is by any means, for example, automated means, wherein changes in physical properties of the sample are quantifiable.

The methods of this invention comprise correlating the detected value with one reflective of the concentration of nucleic acid in the bodily fluid sample. In some embodiments, the detected changes are quantified and correlated with the concentration of nucleic acids, for example as described herein in Examples 1-3. In some embodiments, the detectable moiety is fluorescent, and fluorescence is measured quantitatively in a fluorimeter, and the values obtained for a particular sample are compared to a series of standards, whose nucleic acid concentration is known. According to this aspect and in one embodiment, fluorescence of the standards is determined, under identical conditions as those applied for the sample. According to this aspect and in one embodiment, the sample fluorescence is determined, and the nucleic acid concentration is derived, based on comparability of the fluorescence for known nucleic acid concentrations of the series of standards tested. In some embodiments, a standard curve is derived for the nucleic acid concentration of the standards plotted as a function of fluorescence obtained, and thereby sample concentrations can be determined.

In one embodiment, the method is conducted in parallel to mixing a second bodily fluid sample obtained from a second subject, and said correlating results in said value representing a standard for said method. According to this aspect, and in one embodiment, a first sample nucleic acid is being determined relative to a second sample, whose concentration is not necessarily known, but the status of the source for the nucleic acid material from the second sample serves as a negative standard for the first sample, such that quantification, for example, fluorescence levels, if significantly lower than those obtained from the second sample serve as a diagnostic or prognostic indicator for the subject from whom the first sample was obtained, for example, no disease, or for example, desirable response to therapy, and others as will be appreciated by one skilled in the art. Similarly, in some embodiments, if the quantification, for example, fluorescence levels, yields values significantly higher than those obtained from the second sample serve as a diagnostic or prognostic indicator for the subject from whom the first sample was obtained, for example, presence of disease, or for example, exacerbation of disease, or for example, poor response to therapy, and others as will be appreciated by one skilled in the art. In some embodiments, a series of standards is generated representing severity of a disease or condition, such that increasing values obtained for fluorescence represents discrete stages in disease pathogenesis, for example cancer staging values, and evaluation of a particular sample under identical conditions, in comparison to the series of standards serves as a diagnostic both for the presence and staging of a cancer, according to this aspect.

It will be appreciated that various standards may be employed, whereby the obtained results from a particular sample, when compared to those obtained for the series of standards will serve as a diagnostic or prognostic indicator, as a function of the quantitative result obtained, and/or as a function of a relative value to those obtained in the series of standards.

In one embodiment, the subject has or is predisposed to a disease or disorder. According to this aspect, and in one embodiment, the subject has a genetic predisposition to a disease or disorder, or in another embodiment, the subject has certain lifestyle risk factors associated with a disease or disorder, or in another embodiment, the subject exhibits phenotypic characteristics or symptoms associated with incidence of a disease or disorder.

In one embodiment, the method further comprises diagnosing the presence of said disease or disorder based on said value obtained.

In one embodiment, the method further comprises predicting the severity of said disease or disorder based on said value obtained.

In one embodiment, the method further comprises assessing response of a subject to treatment of said disease or disorder, based on said value obtained.

In one embodiment, the disease or disorder comprises a tissue injury, infection, inflammatory response, neoplasia or preneoplasia. In one embodiment, the tissue injury comprises myocardial infarction. In one embodiment, the disease or disorder comprises sepsis.

In some embodiments, this invention provides a method to determine the presence or absence of a medical condition such as inflammatory diseases or cell proliferative diseases, for example cancer. The method employs, inter alia retrieval of an individual's sample in form of a biological fluid like blood, serum, urine or other fluids as described herein, and others known in the art. In some embodiments, the method employs determining the amount of free DNA in the sample, with the amount or presence (detectable above a given threshold) of free DNA serving as a diagnostic or prognostic indicator, i.e. from this determination, in some embodiments, the presence or absence or severity of a medical condition can be concluded.

In some embodiments, the methods/kits of this invention enable the prediction of whether an individual suffers from, or is at risk for a particular medical condition. In some embodiments, once alterations in nucleic acid levels are rapidly detected, the nucleic acid is further probed for additional characteristics, which in turn may further elucidate for example, not just the presence of a proliferative disease, but the source, e.g. tissue of the proliferative cells. Such secondary determinations may be conducted by any means known in the art, for example by PCR technology, with probes specific for detecting certain characteristic genes, for example, or for example for detecting certain methylation patterns, or others, as will be appreciated by one skilled in the art. In some embodiments, the assays and materials of this invention may be useful in the determination of damage due to over-exercise in, for example, sportsmen or military personnel. Thus, in one embodiment, the assays and materials of this invention may be applicable in the field of sports medicine, as will be appreciated by the skilled artisan.

The methods of this invention, in some embodiments, involve a biological fluid sample being retrieved from a patient or individual. The retrieval of the said sample may be conducted via any means known to a person skilled in the art. In some embodiments, such retrieval may comprise, inter alia, ventricular puncture, also known as CSF collection, a procedure to obtain a specimen of cerebrospinal fluid (CSF); thoracentesis, referring to inserting a needle between the ribs into the chest cavity, using a local anaesthetic to obtain the pleural effusion fluid; amniocentesis, referring to a procedure performed by inserting a hollow needle through the abdominal wall into the uterus and withdrawing a small amount of fluid from the sac surrounding the fetus, or standard means for blood, urine, sperm or sputum collection, or other means.

In some embodiments, nucleic acid quantification as described herein may take place either immediately after retrieval of the sample or after an unspecified time of storage of said sample.

In some embodiments, the methods/kits of this invention find use in the identification of subjects with abnormal amounts of free nucleic acid, with normality being a function of the absence of a deviance of 10% or more from a value defined as “normal”, in their bodily fluids. In some embodiments, normality is a function of the absence of a deviance of 20% or more from a value defined as “normal”, in their bodily fluids, or in some embodiments, normality is a function of the absence of a deviance of 30% or more from a value defined as “normal”, in their bodily fluids, or in some embodiments, normality is a function of the absence of a deviance of 40% or more from a value defined as “normal”, in their bodily fluids.

In some embodiments, such deviance serves as a diagnostic or prognostic indicator. In some embodiments, the term “diagnostic” and grammatical forms thereof, when referred to herein, refers to the ability to demonstrate an increased likelihood that an individual has a specific condition or conditions. In some embodiments, diagnosis also refers to the ability to demonstrate an increased likelihood that an individual does not have a specific condition. In some embodiments, diagnosis refers to the ability to demonstrate an increased likelihood that an individual has one condition as compared to a second condition. In some embodiments, diagnosis refers to a process whereby there is an increased likelihood that an individual is properly characterized as having a condition (“true positive”) or is properly characterized as not having a condition (“true negative”) while minimizing the likelihood that the individual is improperly characterized with said condition (“false positive”) or improperly characterized as not being afflicted with said condition (“false negative”).

In some embodiments, the term “prognostic” and grammatical forms thereof, when referred to herein, refers to the ability to predict the progression or severity of a disease or condition in an individual. In some embodiments, prognosis also refers to the ability to demonstrate a positive response to therapy or other treatment regimens, for the disease or condition in the subject. In some embodiments, prognosis refers to the ability to predict the presence or diminishment of disease/condition associated symptoms.

In some embodiments, the methods/kits described herein find application in diagnostic, prognostic and research purposes, wherever it is advantageous to determine the relative or absolute amount of nucleic acid in a sample. For example, the methods disclosed herein can be used to diagnose aneuploidies, such as occur in, for example, neoplastic cells and in individuals, e.g., fetuses or postpartum individuals or adults, afflicted with a genetic disorder.

In some embodiments, the methods/kits described herein find application in the diagnosis and/or prognosis of inflammatory diseases in a subject. In some embodiments, such inflammatory disease are, or the presence of inflammation indicates diseases such as adult respiratory distress syndrome (ARDS), allergies, arthritis, asthma, autoimmune diseases (e.g., multiple sclerosis), bronchitis, cancer, cardiovascular disease, chronic obstructive pulmonary disease, Crohn's disease, cystic fibrosis, emphysema, endocarditis, gastritis, graft-versus-host disease, infections (e.g., bacterial, viral and parasitic), inflammatory bowel disease, injuries, ischemia (heart, brain, placental, etc.), multiple organ dysfunction syndrome (multiple organ failure), nephritis, neurodegenerative diseases (e.g., Alzheimer's disease and Parkinson's disease), ophthalmic inflammation, pain, pancreatitis, psoriasis, sepsis, shock, transplant rejections, trauma, ulcers (e.g., gastrointestinal ulcers and ulcerative colitis), and many others.

In some embodiments, the methods/kits described herein find application in the diagnosis and/or prognosis of cell proliferative diseases in a subject. In some embodiments, such proliferative diseases comprise cancers, including but not limited to biliary tract cancer; brain cancer, including glioblastomas and medelloblastomes; breast cancer; cervical cancer; choriocarcinoma; colon cancer; endometrial cancer; esophogeal cancer; gastric cancer; hematological neoplasms, including acute lymphocytic and myelogeneous leukemia, multiple myeloma, AIDS associates leukemias and adult T-cell leukemia lymphoma; intraepithelial neoplasms, including Bowen's disease and Paget's disease; liver cancer; prostate cancer, lung cancer; lymphomas, including Hodgkin's disease and lymphozytic lymphomas; neuroblastomas; oral cancer, including squamous cell carcinoma; ovarian cancer, including those arising from epithelial cells, stromal cells, germ cells and mesenchymal cells; pancreas cancer; rectal cancer; sarcomas, including leiomyosarcoma, rhabdomyosarcoma, liposarcoma, fibrosarcoma and osteosarcoma; skin cancer, including melanoma, Kaposi's sarcoma, basocellular cancer and squamous cell cancer; testicular cancer, including germinal tumors (seminoma, non-seminoma (teratomas, choriocarcinomas)), stromal tumors and germ cell tumors; thyroid cancer, including thyroid adenocarcinoma and medullar carcinoma; renal cancer including adenocarcinoma and Wilms tumor and others.

In some embodiments, the invention provides a method of quantifying the in vitro nucleic acid concentration in a tissue culture fluid, the method comprising the steps of:
a) mixing a tissue culture fluid sample with a detectable nucleic acid intercalating agent, wherein said mixing is conducted in the absence of prior nucleic acid extraction;
b) detecting said moiety; and
c) correlating detection of said moiety with a value reflective of the concentration of nucleic acid in said tissue culture fluid sample.

In some embodiments, the method comprises mixing serially diluted tissue culture fluids. In some embodiments, the detectable nucleic acid intercalating agent comprises a detectable moiety, which in some embodiments, is fluorescent and in some embodiments, the detecting is conducted with the use of a fluorimeter. In some embodiments, the detectable nucleic acid intercalating agent comprises SYBR Gold© or SYBR Green©.

In some embodiments, the method is conducted in parallel to mixing a second tissue fluid sample obtained from a source subjected to an alternative culture condition than that of said first tissue culture fluid sample.

In some embodiments, correlating includes assigning a value to said tissue fluid sample based on the comparative detection with that obtained for said second fluid sample.

In some embodiments, the method further comprises mixing a second tissue culture fluid sample comprising a known concentration of nucleic acid with said detectable agent and correlating detection with a value equal to said known concentration.

In some embodiments, this invention provides a method of quantifying the residual nucleic acid concentration in a recombinant protein bioreactor fluid, the method comprising the steps of:
a) mixing a fluid sample obtained from a bioreactor for the preparation of recombinant proteins with a detectable nucleic acid intercalating agent, wherein said mixing is conducted in the absence of prior nucleic acid extraction;
b) detecting said moiety; and
c) correlating detection of said moiety with a value reflective of the concentration of nucleic acid in said fluid sample.

In some embodiments, the detectable nucleic acid intercalating agent comprises a detectable moiety, which in some embodiments is fluorescent, and in some embodiments, the detecting is conducted with the use of a fluorimeter. In some embodiments, the detectable nucleic acid intercalating agent comprises SYBR Gold© or SYBR Green©.

In some embodiments, the method indicates bioreactor efficiency.

In one embodiment, this invention provides a kit for the quantification of the nucleic acid concentration of a bodily fluid of a subject, said kit comprising:
a) a detectable nucleic acid intercalating agent;
b) a diluent; and
c) a series of solutions comprising nucleic acid samples in said diluent, wherein the concentration of each of the nucleic acid samples in said series is known;
   whereby a bodily fluid sample is mixed with said detectable nucleic acid intercalating agent in parallel to mixing said agent with said series, and detection of said agent in said series serves as a standard for arriving at a value reflective of the concentration of nucleic acid in said bodily fluid sample.

It is to be understood that the intercalating agents, series of solutions comprising standards, etc. may comprise any embodiment thereof as described herein, and any others appropriate, as will be appreciated by the skilled artisan.

The diluent described herein may be any suitable solvent or solution, which serves to dilute the sample as desired, and wherein the properties of the diluent do not interfere with the detection and/or quantification of the nucleic acid in the sample. In some embodiments, the diluent is any suitable buffer, or solution, for example, physiological saline, or for example, phosphate buffered saline, and others as will be appreciated by the skilled artisan.

In one embodiment, the kit optionally comprises a container suitable for accommodating the series of solutions and said bodily fluid sample and wherein the container may by applied to a fluorimeter. In some embodiments, the methods/kits of this invention lend themselves to automation, and standard assay dishes and plates, for example 96 well plates commonly sold by commercial vendors are suitable for use. In some embodiments, the apparatus utilized for the detection as described herein, will accommodate such containers readily, further adding to the ease and cost-effectiveness of the kits/methods described herein.

The kits may be formatted for use in a diagnostic apparatus (e.g., an automated analyzer) or can be self-contained (e.g., for a point-of-care diagnostic).

In one embodiment, the kit comprises a container suitable for the assay of urine, blood or a component thereof, lavage fluid or a combination thereof. Biological fluids often represent a hazard for the technician assaying the same, and various means have been developed to minimize exposure and thereby risk to the technician performing the assay, for example transfer with plastic, non-sharp transfer means of the fluid to the assay container, seals for such containers, etc. In one embodiment of this invention, as the assay provides for rapid quanitification, kits are particularly constructed such that as many safety precautions as possible are employed for use with the sample fluids to minimize risk while maximizing speed in effecting the methods of this invention.

Kits for determining the quantities of nucleic acids will comprise one or more containers holding reagents useful for performing the assays, including, for example, containers holding standards and intercalating agents. Suitable containers for the reagents of the kit include bottles, vials, test tubes and microtiter plates. Also, reagents (e.g., intercalating) can be incorporated into or onto substrates, test strips (made of, e.g., filter paper, glass, metal, plastics or gels) and other devices suitable for performing the assay. Instructions for performing one or more assays for quantifying nucleic acid will be provided with the kits (e.g., the instructions can be provided in the same package holding some or all of the reagents or can be provided in separate documentation). The kit may also contain other materials which are known in the art and which may be desirable from a commercial and user standpoint, such as buffers, enzyme substrates, diluents, standards, etc. Finally, the kit may include containers, such as empty containers for performing the assay, for collecting, diluting and/or measuring a body fluid, and/or for diluting reagents, etc.

Kits for diagnosing diseases or conditions described herein will comprise one or more containers holding reagents useful for the same, including secondary assay materials/reagents for further identification, once the initial finding of altered nucleic acid concentration is ascertained. Such kits may be two-part kits, each part providing the reagents and other materials for performing one of the assays. Instructions for performing each of assays will be provided with the kits (e.g., the instructions can be provided in the same package holding a two-part kit, can be provided in each of the packages holding the separate kits, or can be provided in separate documentation).

The kit may comprise a container holding a color-producing material (i.e., a material capable of undergoing a color-producing reaction when contacted with the intercalating agent). Such a kit may further comprise a container for collection of a body fluid (such as a syringe or a plastic or paper cup), an instrument for measuring the body fluid (such as a dropper, a pipette or a micropipette) and either a color comparison chart or containers holding standards comprising known amounts of nucleic acid.

It will be understood by those skilled in the art that various changes in form and details may be made therein without departing from the spirit and scope of the invention as set forth in the appended claims. Those skilled in the art will recognize, or be able to ascertain using no more than routine experimentation, many equivalents to the specific embodiments of the invention described herein. Such equivalents are intended to be encompassed in the scope of the claims.

In the claims articles such as “a,”, “an” and “the” mean one or more than one unless indicated to the contrary or otherwise evident from the context. Claims or descriptions that include “or” or “and/or” between members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides, in various embodiments, all variations, combinations, and permutations in which one or more limitations, elements, clauses, descriptive terms, etc., from one or more of the listed claims is introduced into another claim dependent on the same base claim unless otherwise indicated or unless it would be evident to one of ordinary skill in the art that a contradiction or inconsistency would arise. Where elements are presented as lists, e.g. in Markush group format or the like, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in haec verba herein. Certain claims are presented in dependent form for the sake of convenience, but Applicant reserves the right to rewrite any dependent claim in independent format to include the elements or limitations of the independent claim and any other claim(s) on which such claim depends, and such rewritten claim is to be considered equivalent in all respects to the dependent claim in whatever form it is in (either amended or unamended) prior to being rewritten in independent format.

### EXAMPLES

### Materials and Methods

CFD is detected with the present assay directly in biologic fluids. SYBR® Gold Nucleic Acid Gel Stain, (Invitrogen, Paisley, UK) was diluted first at 1:1000 in DMSO and then at 1:8 in phosphate buffered saline (PBS, Biological Industries, Beth Haemek, Israel). 10 μl of DNA solutions were applied to a black 96 wells plate (Greiner Bio-One, Frickenhausen, Germany). 40 μl of diluted SYBR® Gold was added to each well (final dilution 1:10,000) and fluorescence was measured with a 96 well fluorometer (Spectrafluor Plus, Tecan, Durham, N.C.) at an emission wavelength of 535 nm and an excitation wavelength of 485 nm. EvaGreen (PCR-352, Jena Bioscience, Jena Germany) was used to stain DNA standards (PBS+2% BSA diluted in 96 well plates) at a 1:1000 dilution and fluorescence was measured at 535, same conditions as SybrGold).

### Background Fluorescence of Serum

For assessment of background reading and to establish the optimal serum concentration in DNA standard solutions, we used pooled human serum from ten healthy donors. Sera were preincubated at 37° C. overnight with either RNase (100U, Sigma-Aldrich) or for 5 hours with DNase (500 U, 5-PRIME, Gaithersburg, USA). DNase was inactivated by 20 mM EDTA prior to addition of DNA standards.

For assessment of background and quenching of serum, serum was diluted with PBS to various concentrations (0, 10, 20, 30 and 40% serum, respectively); same amount of salmon sperm DNA was added to all solutions resulting in a final DNA concentration of 1140 ng/ml. Assay was performed in triplicates. Serum solutions at same concentrations not containing DNA were used to determine background fluorescence.

### DNA Standards

For the fluorometric assay, standards were prepared with commercial Salmon sperm DNA (Sigma-Aldrich, Rehovot, Israel). For comparison with the conventional QPCR assay, human DNA was extracted from blood leukocytes using QIAamp Blood Kit (Qiagene, Hilden, Germany) according to the manufacturer's protocol. Concentrations of DNA used for the standard curves were determined by UV absorbance at 260 nm using a Nano-Drop spectrophotometer (Thermo Fisher Scientific, Wilmington, Del. USA).

### Biological Fluids

To assess the dose response of the assay in different fluids, salmon sperm DNA was diluted at various concentrations in four different fluids: A. 20% solution of pooled serum from 10 healthy donors in PBS. B. 2% solution of bovine serum albumin (BSA, Biological Industries, Beth Haemek, Israel) in PBS. C. Heparinized fresh whole blood from a healthy donor and D. pooled urine from 10 healthy donors buffered to pH 7.4 with 10 mM HEPES (Biological Industries).

### Effect of Storage Conditions

Whole Blood: Refrigeration vs. Room Temperature

To assess the effect of storage temperature on the assay, eight whole blood samples were collected from healthy volunteers into commercial gel tubes using the BD Vacutainer® system (Becton, Dickinson and Company, Plymouth, UK). Centrifugation was postponed; 5 tubes from each donor were stored for 0.5, 1.4, 5 and 24 hours at room temperature (RT) and 3 tubes were stored for 0.5, 4, 24 hours at 4° C. At respective time points, tubes were centrifuged, sera were collected and assayed for DNA by the direct SYBR® Gold assay.

### Serum: Room Temperature

In a further experiment, aliquots of one human serum were incubated at RT and each aliquot was assayed at different time points. In addition, 10 random sera from our serum bank have been grouped according to their DNA level as measured by the direct SYBR® Gold assay into one of three groups: low, elevated and high range of DNA level. (low range group: 580, 460, 475 ng/ml; elevated range group: 2410, 2180, 2440, 2005 ng/ml; high range group: 3515, 3975, 3570 ng/ml). Sera have been thawed and aliquots were incubated for 24 hrs at RT and compared for their DNA levels with their corresponding aliquots which were kept at −20° C.

### Serum: Repeated Freezing and Thawing

Aliquots of the same 10 sera were frozen and thawed 5 times and compared for their DNA levels with their corresponding aliquots which were kept at −20° C. and thawed only once.

### Within-Day Variation

Intra-day variation of the assay was assessed by comparing readings of 12 assays done independently on separate plates at different times over one working day. In each assay, duplicates of 3 sera with low, elevated and high DNA concentrations (197, 1096 and 4107 ng/ml, respectively) were analyzed.

### Day to Day Variation

Day to day variation was assessed by comparing readings of aliquots from same sera on 4 different days. A total of 12 aliquots were analyzed in duplicates from three donors with low, elevated and high DNA concentrations (383, 1152 and 2735 ng/ml, respectively).

Comparison with Conventional CFD Assay

We also compared the direct SYBR® Gold assay with a conventional method of CFD assay as follows: Standards of human DNA were analyzed by quantitative real-time PCR (QPCR) amplification of the β-globin gene. The amplification mixture contained: 7 μl of DNA samples or human DNA standards (15-1000 ng/ml) in QIAamp elution buffer 2 μl of each primer (20 μM), 10 μl of ABsoluteBlue QPCR SYBR Mix Rox (ABgene, Surrey, UK) and water to a final volume of 20 μl. The primers and QPCR conditions of the human β-globin gene have been previously described (Jung M, Klotzek S, Lewandowski M, Fleischhacker M, Jung K. Changes in concentration of DNA in serum and plasma during storage of blood samples. Clin Chem 2003; 49:1028-9): Forward primer: 5′-ACACAACTGTGTTCACTAGC-3′ (SEQ ID NO: 1), Reverse primer: 5′-CAACTTCATCCACGTTCACC-3′ (SEQ ID NO: 2).

The reaction was carried out in a Rotor-Gene real time PCR machine (Corbett-Research, Northlake, Australia). Cycle conditions were: initial activation step at 95° C. for 15 min, followed by 45 cycles of denaturation at 95° C. for 15 s, annealing at 56° C. for 20 s, and extension at 72° C. for 15 s. In parallel, the human standards were diluted in 20% DNase-treated pooled sera and assayed by the direct SYBR® gold assay. Correlation between the direct SYBR® Gold assay and the QPCR assay was assessed by the Spearman rank test.

### Reference Values

Reference value*s were established by analysis of sera from 47 healthy volunteers. The volunteers were mostly students which declared to be healthy and without chronic disease. The cohort consisted of 22 females and 25 males with an average age of 26.3±4.7 years. Three samples were excluded from the reference group: two of them because of hemolysis and one because the donor was diagnosed with acute infectious mononucleosis

Culture Media DNA after Cell Lysis

Cultured fibroblast cells (L-cells) were seeded at various numbers in triplicates (0, 40, 60, 80, 100, 120, 150 and 200×103 cells/well) in 24 well plates with 1 ml of DMEM medium containing 10% fetal calf serum (Biological Industries). Cell lysis was induced with a detergent (0.1% NP40) and gentle agitation for 30 minutes. Supernatants were collected and assayed for DNA concentration by the direct SYBR® Gold assay. In addition, LDH activity was assayed in the supernatant using a commercial kit (BioVision, Mountain View, Calif., USA) according to the manufacturer's protocol.

### Statistics

Statistic analysis was performed with GraphPad Prism® software (edition 4.01), Statistical significance was determined by t-test or analysis of variance. Significance of correlation was analyzed by Pearson-r test. A p-value <0.05 was considered significant.

Peritoneal lavage fluid and blood were collected from CD1 female mice aged 10 to 12 weeks (Harlan, Jerusalem, Israel) suffering from peritonitis 24 hours following induction by intraperitoneal E. coli inoculation with a sub-lethal dose (3.6×10⁹ CFU) as well as from age- and weight-matched controls. Serum was collected from human patients being hospitalized for a myocardial infarction or suspected thereto. Serum was collected from healthy donors, as well, serving as controls. Serum/lavage fluid was diluted 1:5 in PBS, and applied to 96 well tissue culture dishes.

Serially diluted (PBS) samples of known quantities of salmon DNA in 20% normal pooled human sera or whole blood were similarly applied to tissue culture dishes with 1:10000 dye.

SYBR Gold or SYBR green was added to each well such that the final dilution of the fluorochrome was 1:10,000. Fluorescence was assessed in a Fluorimeter, with excitation at 485 nm, emission at 535 nm.

### Example 1

### Rapid DNA Quantification in Biological Fluid

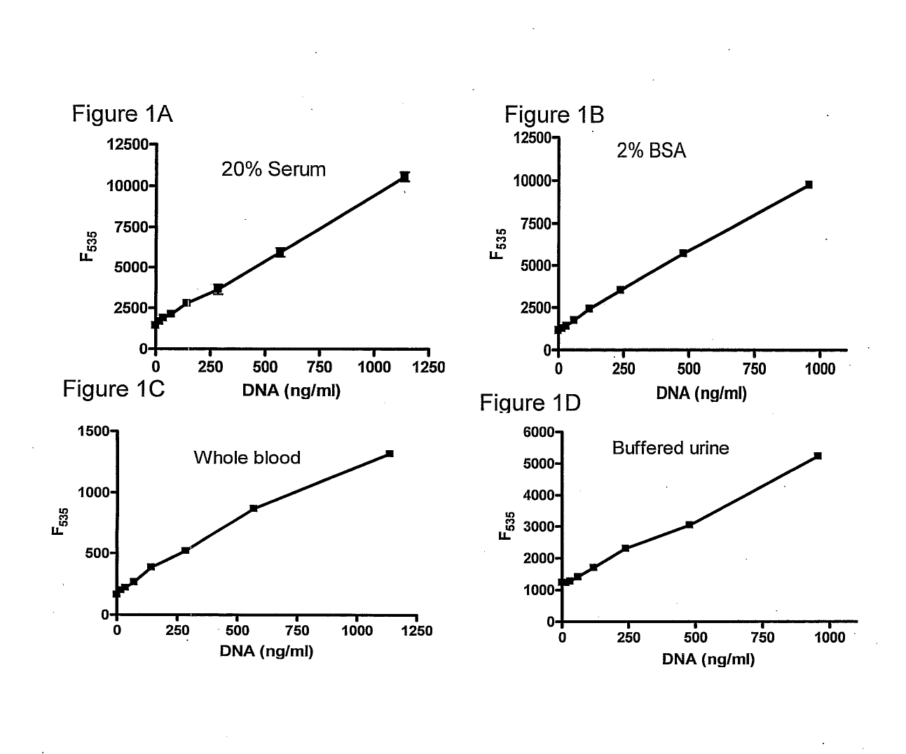

In order to determine whether rapid DNA quantification was obtainable using a DNA intercalating moiety, serum containing known dilutions of DNA was mixed with SYBR Gold, and fluorescence was determined (FIG. 1A). Concentrations of as little as 100 ng/ml of DNA were detected. Linearity was observed when 2% BSA, whole blood or buffered urine containing known dilutions of DNA were mixed with SYBR Gold as well (FIGS. 1B, 1C and 1D).

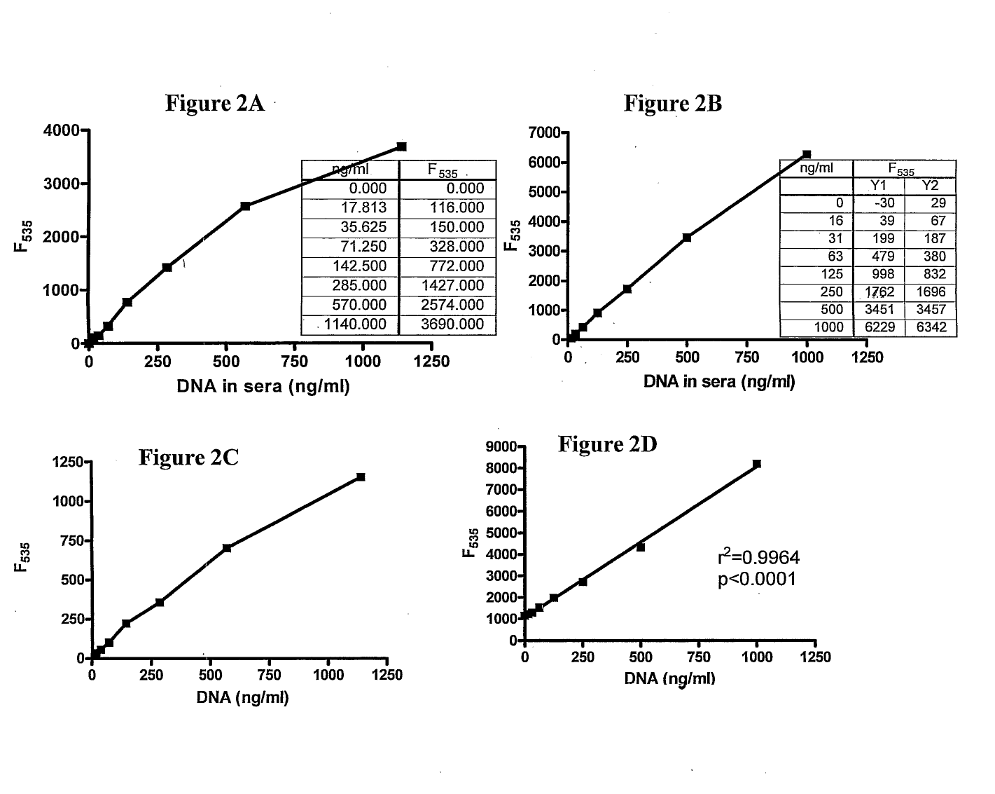

Similarly, fluorescence of side-by-side comparisons of serially diluted salmon DNA in 20% normal pooled human sera showed comparable detection, when two different intercalating agents were utilized (FIGS. 2A and B). Detection using SYBR gold in whole blood yielded comparable results (FIG. 2C) as did detection using EvaGreen (FIG. 2D).

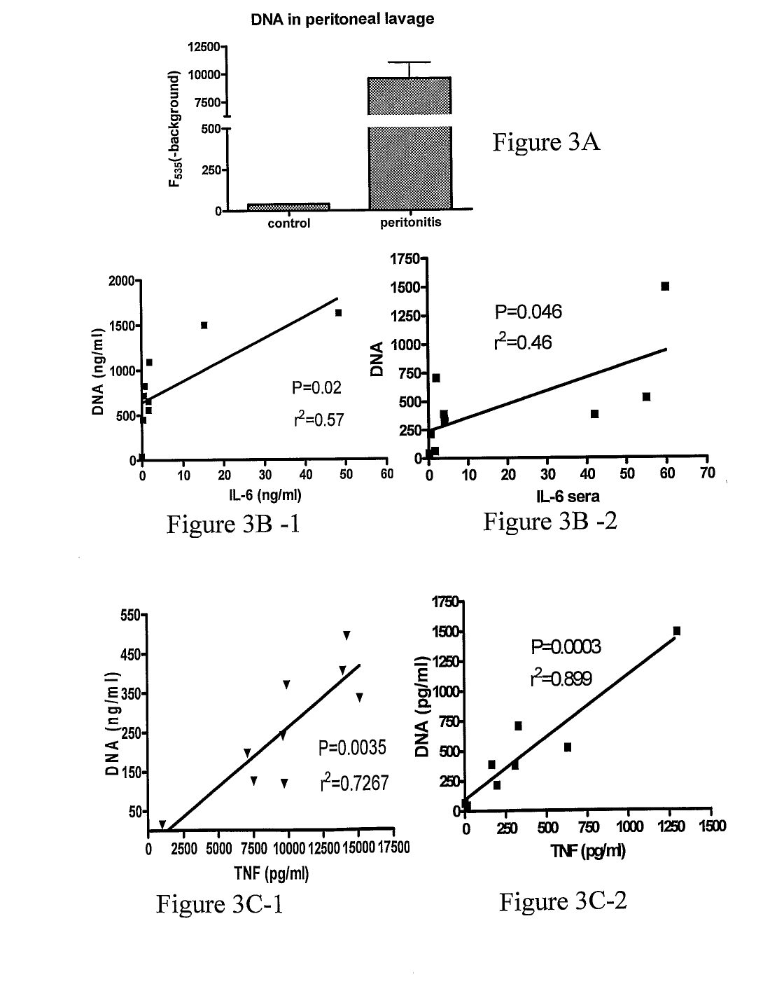

In order to determine whether rapid DNA quantification was obtainable in biological samples, peritoneal fluid obtained by lavage of mice undergoing peritonitis induced by intra-peritoneal E. coli injection was mixed with the intercalating agent, without prior DNA extraction (FIG. 3A). Mice undergoing E. coli-induced inflammatory responses demonstrated significantly greater amounts of free DNA in biological fluids as compared to controls.

Moreover, total DNA concentration correlated well with the presence of IL-6 and TNF induction in lavage fluid and in serum (FIGS. 3B and 3C, respectively).

### Example 2

### Rapid DNA Quantification in Human Sera

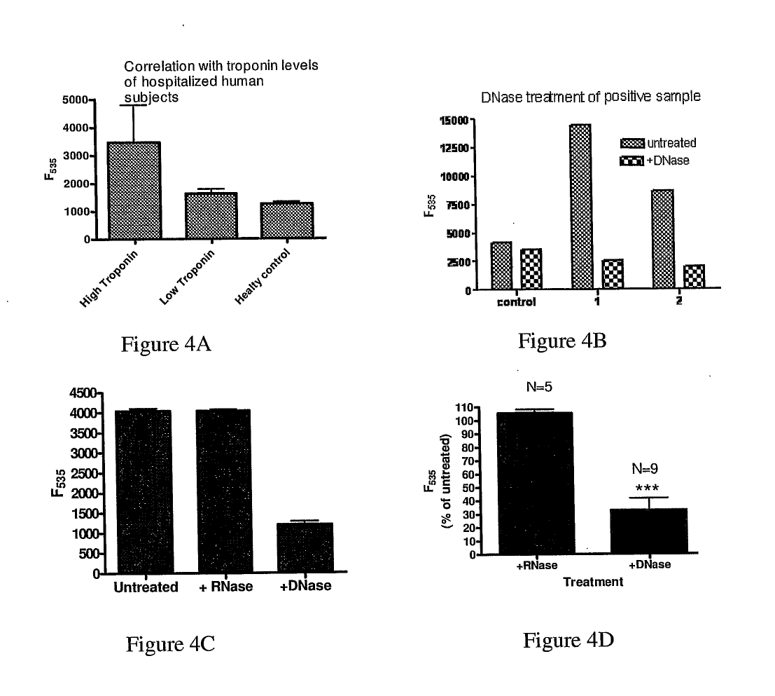

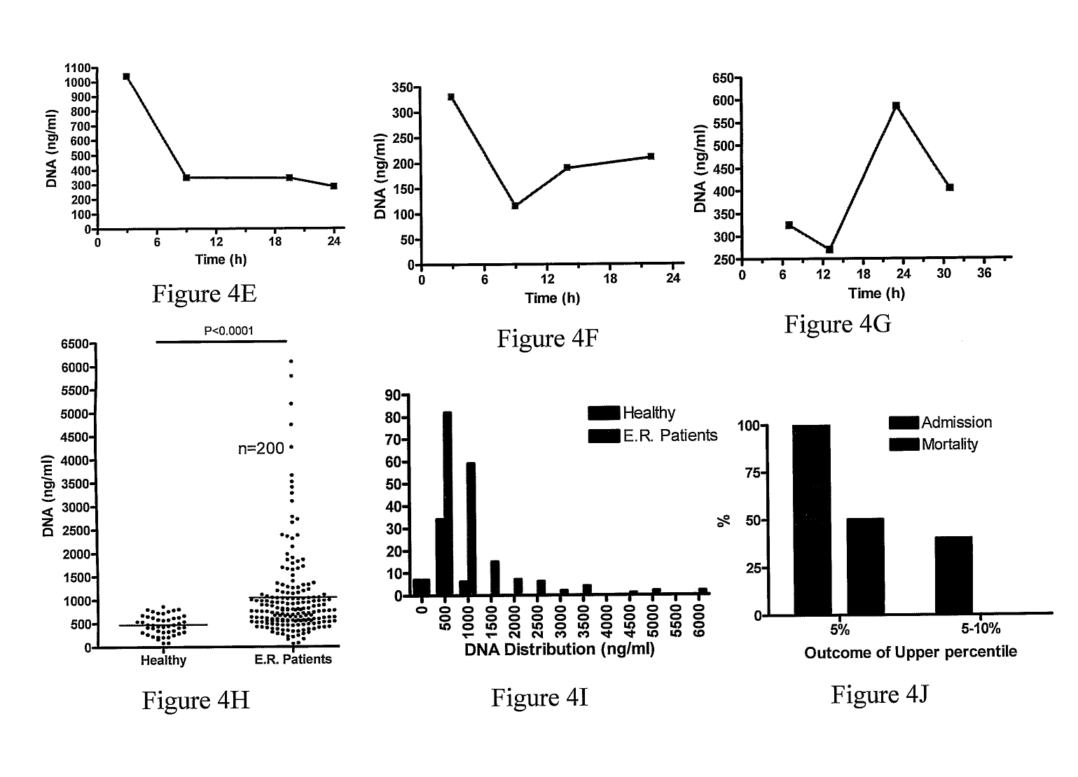

Example 1 demonstrated rapid DNA quantification in biological fluids including sera of mice, thus it was of interest to determine whether such assay would be useful as an indicator of DNA concentration in human sera. Toward this end, serum was collected from Human subjects arriving at the Emergency room with suspected myocardial infarction. FIG. 4A demonstrates that serum troponin levels (a protein released from cardiac muscle following an ischemic event) correlate well with DNA levels detected by the assay as described herein, again without necessity for DNA extraction prior to quantification. Treatment of the serum sample with DNase abolished detection indicating the specificity of the assay (FIG. 4B).

FIGS. 4C and 4D demonstrate the specificity of the assay for DNA and not other nucleic acid, as addition of RNase did not abrogate detection (FIGS. 4C and 4D). Patient samples were treated with RNase or DNase, and the percent fluorescence reduction of 5 different samples treated with RNase (n=5) and 9 with DNase were compared before (100%) and after treatment.

FIGS. 4E-G show quantification of DNA in samples from hospitalized patients with acute myocardial infarction (MI) at different hours from arrival to emergency room. FIGS. 4H-J depict the DNA levels (H), Distribution, (I) and patients outcome (J) of 200 subjects who were evaluated in a Hospital Emergency Room. A trend was evident that subjects who visited a Hospital Emergency Room had higher serum DNA levels as compared to healthy subjects (4H). FIG. 4J demonstrates the usefulness of the assay as a predictor for mortality, with an almost 50% mortality rate in subjects representing the upper 5% of subjects assay demonstrating high DNA concentration.

Thus a rapid, cost-effective and easy to use assay for DNA quanitification in biological fluids has herein been developed, which does not necessitate prior DNA extraction.

### Example 3

### Rapid DNA Quantification in the Absence of Prior DNA Extraction

In order to delineate whether the sensitivity of detection is compromised without prior DNA extraction, side-by-side DNA quantification was conducted on samples in which DNA was subjected to a prior extraction step, or not (FIG. 5).

Panel A describes the dose-dependent fluorescence of DNA samples isolated from whole blood and extracted, per the QIAamp DNA blood Kit (Qiagen). DNA was extracted from healthy donor leukocytes, and suspended in buffer with a final concentration of 20% normal human serum, which does not appreciably differ from Panel C, showing direct DNA assay, without prior extraction.

Panel B describes the correlation of DNA samples isolated from whole blood and extracted, per the QIAamp DNA blood Kit (Qiagen) with β-globin copy number. Panel D shows the linear correlation of human DNA standards quantified in parallel by the conventional method and by SYBR gold.

Surprisingly, prior extraction of the DNA samples did not result in appreciably different results regarding DNA quantification, and moreover, detection may be somewhat compromised by prior extraction. These data support the fact that the rapid DNA quantification assay of this invention is highly specific, cost-effective, and non-labor intensive.

### Example 4

### Rapid DNA Quantification Assay Stability

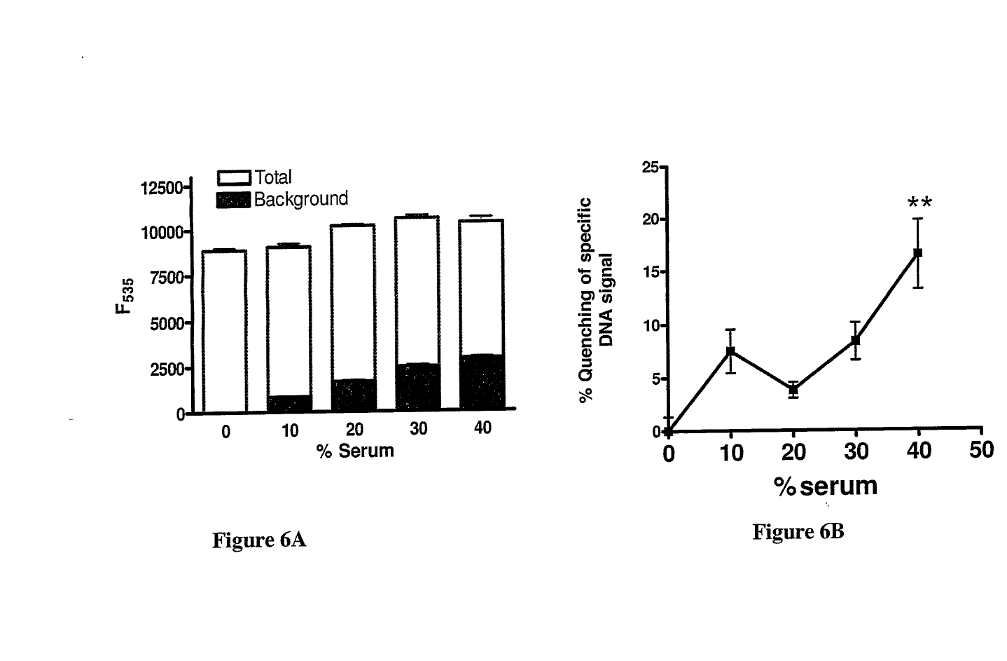

To determine whether test results were maintained stable over time, serum protein fluorescence and quenching was determined (FIG. 6). Pooled human serum was preincubated with DNase and diluted with PBS to various concentrations; same amount of salmon sperm DNA was added to all solutions resulting in a final concentration of 1140 ng/ml. Serum solutions at same concentrations not containing DNA were used to determine background fluorescence. A. Total and background fluorescence of serum solutions B. Calculated % quenching of the specific DNA signal [100−100×(Total F535−Background F535)/total F535]. Assay was performed in triplicates, ** indicates p<0.01 comparing serum solution to PBS without serum.

FIG. 6A demonstrates the sensitivity of the assay in detecting DNA concentration, in comparison to background fluorescence, and 6B indicates lack of appreciable quenching of the specific signal, with increasing serum concentrations, even up to serum levels of 30%.

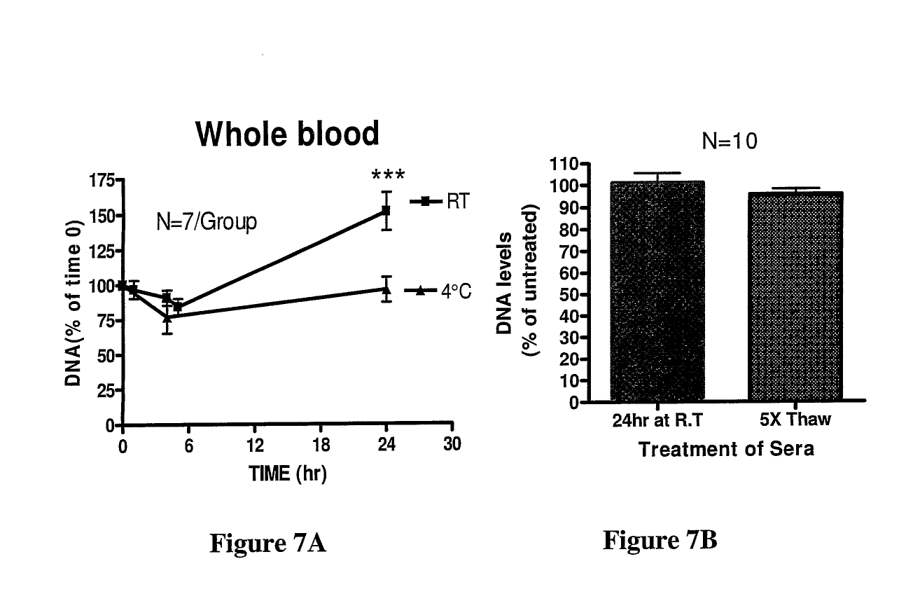

To determine whether the DNA in the test samples may be stable over time, whole blood samples were kept at room temperature or at 4° C. over time (FIG. 7A) with minimal differences observed in quantification of DNA for up to 6 hours in either case. Similarly, repeat freeze-thaw cycles (five) of sera did not appreciably alter DNA stability and thereby influence quantification, in comparison to samples at room temperature (FIG. 7B).

### Example 5

### Rapid DNA Quantification Assay Standardization

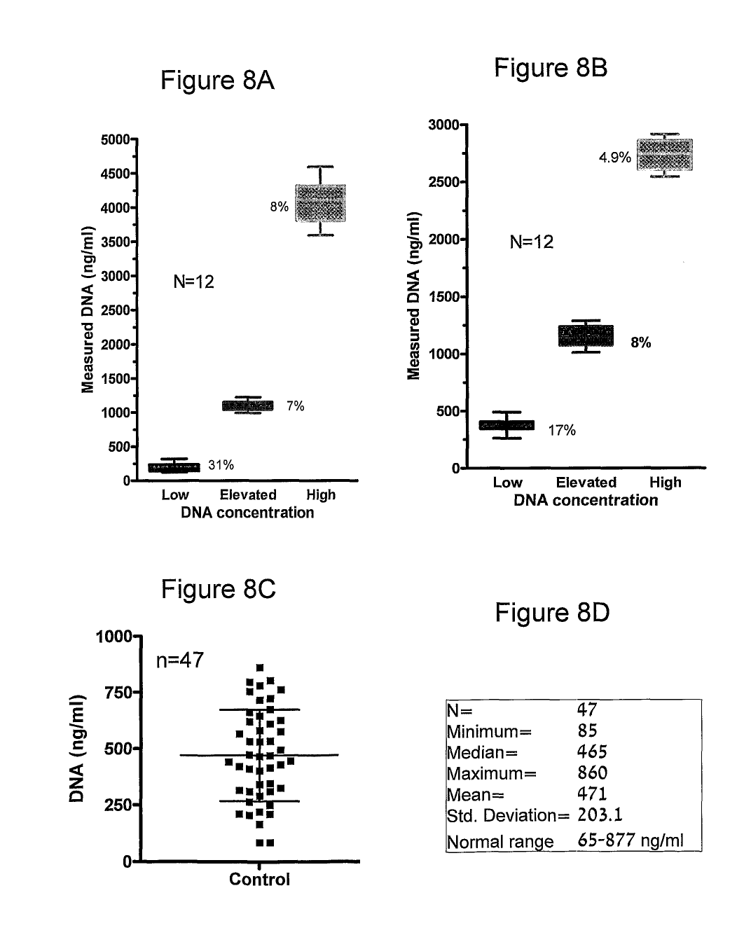

FIG. 8 demonstrates intra & inter assay variation. Intra-day and Intra-assay variation: To assess the variation of the assay, three patients sera were used containing normal, elevated and high DNA concentrations (197, 1096 and 4107 ng/ml, respectively) 8A. Intra-day variation of the assay was assessed by comparing readings of 12 assays of each sample in duplicates done independently on separate plates at different times over one working day. 8B. Day to day variation was assessed by comparing readings of 12 aliquots of each sample. Aliquots have been frozen and assayed on different days. For this assay, serum of three donors was used with low, elevated and high DNA concentrations (383, 1152 and 2735 ng/ml, respectively). Median value of the assays is indicated by the line inside the box. The Box indicates the distribution of 50% of the results and the bar above and below the box indicates 25% of the data.

When assay of the samples was repeated on different days, minimal variability between obtained results occurred, regardless of whether the DNA concentration in the serum was elevated or high (FIG. 8B). Some variation was observed, however, when samples containing a low DNA concentration were assayed. The cell free DNA assay range was evaluated in healthy donors of 47 consisted of 22 women and 25 man with an average age of 26.3±4.7 years. The volunteers were declared to be healthy and with no chronic disease. Subjects demonstrated some variability in terms of typical DNA concentrations and an average level of 471±203 ng/ml, was found. Thus the normal range (mean±2 std) is between 65-877 ng/ml in the samples tested.

### Example 6

### Rapid DNA Quantification as a Diagnostic and Prognostic Assay

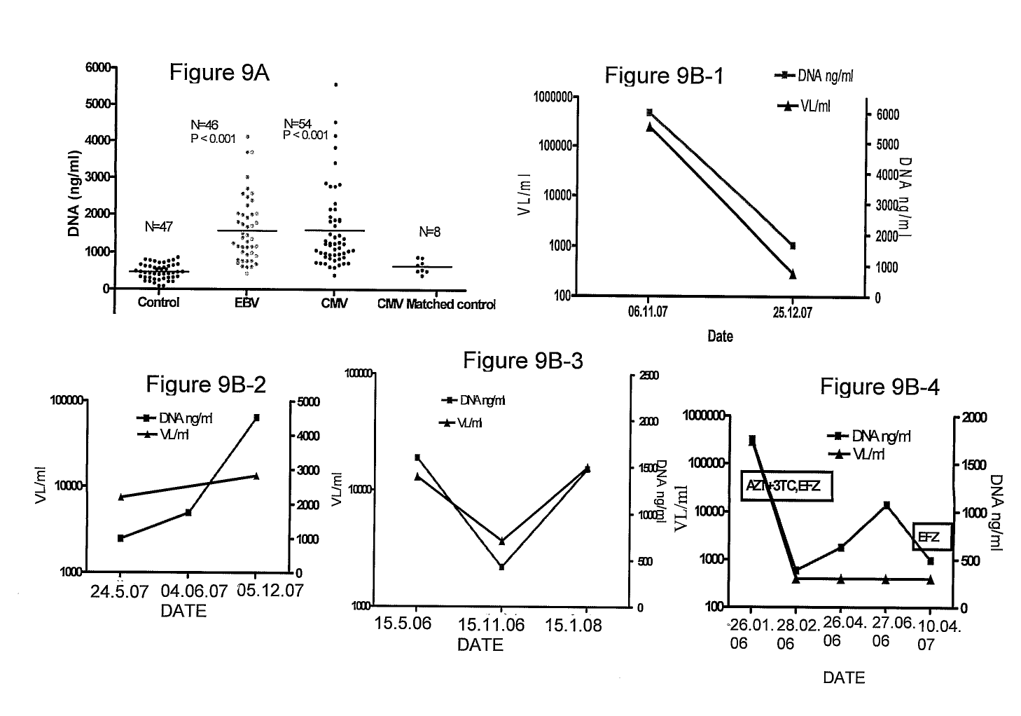

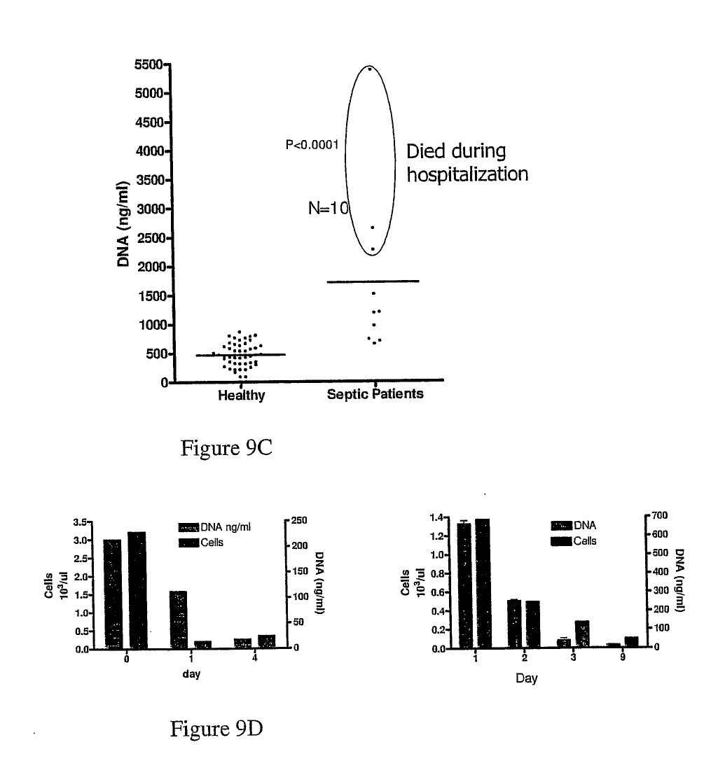

Example 2 demonstrates the potential usefulness of the rapid DNA quantification assays of this invention as a diagnostic and prognostic assay. To extend these studies, the assay was utilized to determine whether it can serve as an indicator of infection and potentially an indicator of severity of infection. FIG. 9A demonstrates that greater DNA concentration may be detected in serum collected from subjects with active EBV and CMV viral infection, as opposed to controls. FIG. 9B demonstrates that DNA quantification correlated well with viral load in HIV infected patients.

FIG. 9C demonstrates that in sepsis, as well, a clear increase in circulating DNA levels is observed, and that mortality correlated with highly elevated DNA levels. FIG. 9D demonstrated that in subjects with active peritonitis, leukocyte number in the peritoneum correlated well with DNA concentration.

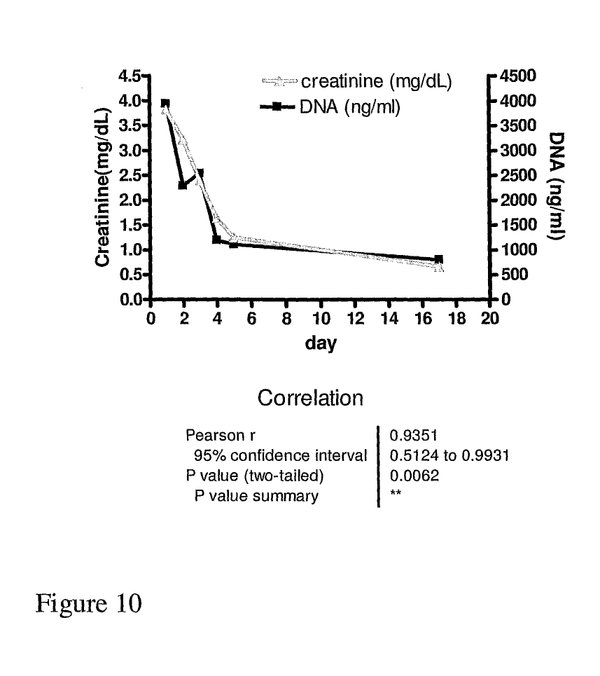

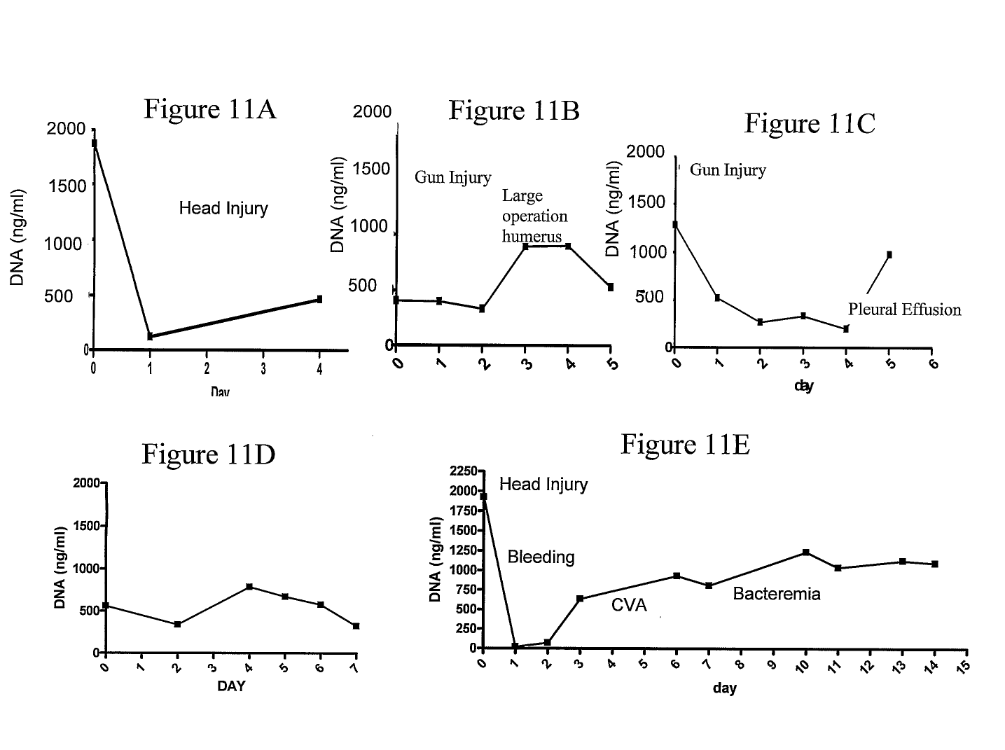

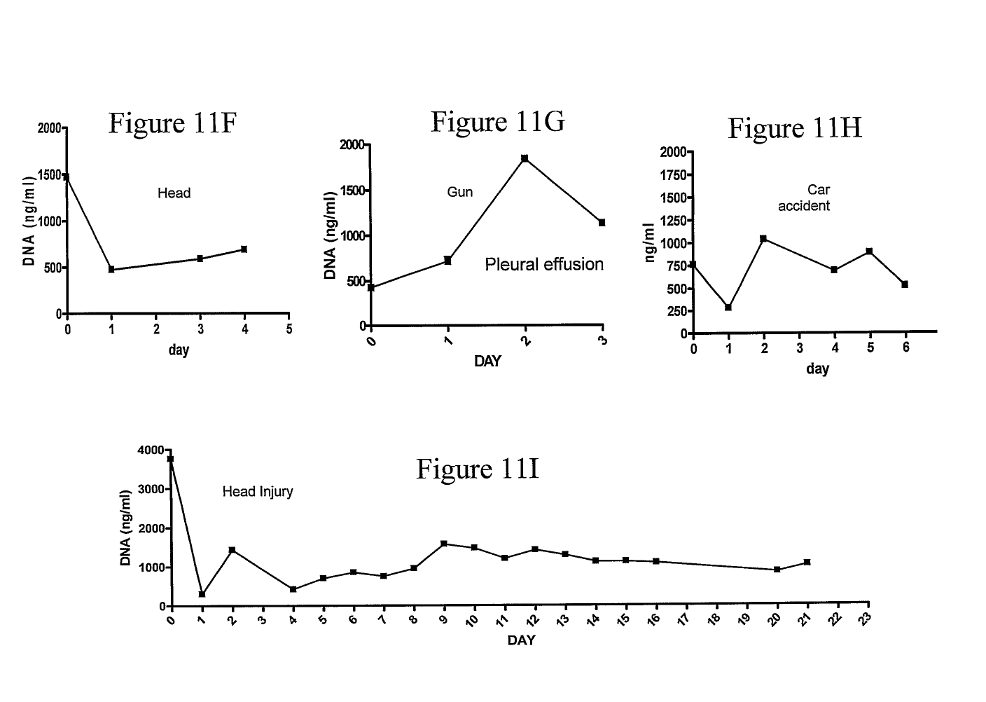

Other clinical conditions may similarly be identified via the use of the rapid DNA quantification assays of this invention. For example, recovery of grafted kidney function may be assessed, with DNA levels correlating well with creatinine levels, in patients following treatment with immunosuppressive drugs (FIG. 10). Circulating DNA levels in trauma patients rise as well (FIG. 11), with additional increases as a function of clinical complications arising in each subject, for example, the presence of pleural effusion in FIG. 11B, or subsequent bacteremia in FIG. 11E.

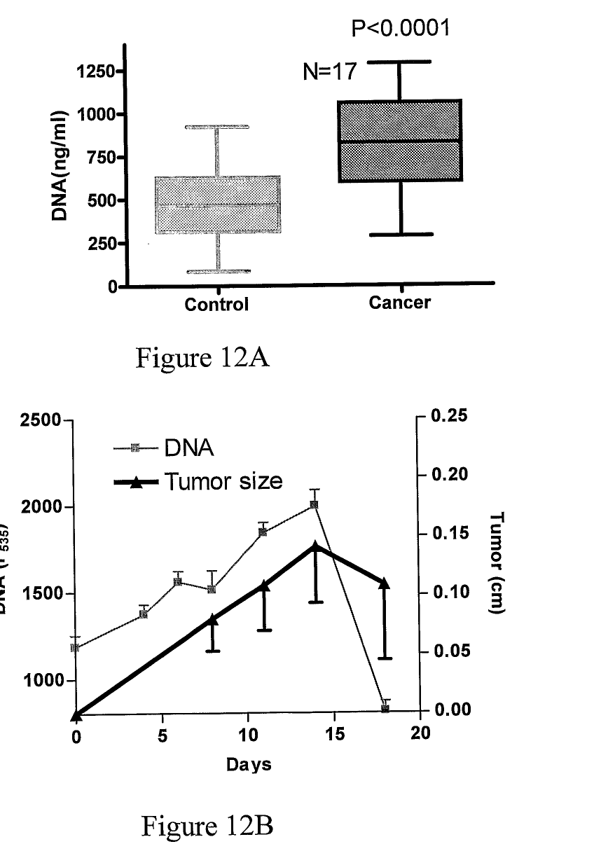

In order to determine whether detection of elevated DNA levels is found in cancer subjects, mice were inoculated intra footpad with an MCA-2 fibrosarcoma cell line with 1.0×10⁶ cells/mouse (N=10). Elevated circulating DNA levels were found to correlate with tumor size (FIG. 12B). CFD levels were elevated in patients with colon dancer, as well, one week before tumor removal (FIG. 12A).

### Example 7

### Rapid DNA Quantification as a Diagnostic and Prognostic Assay

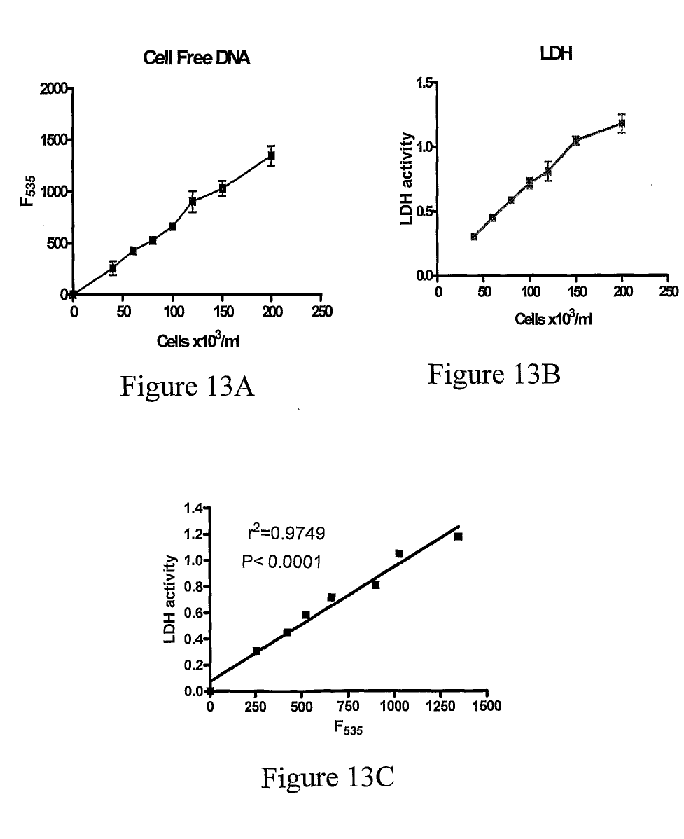

The assays of this invention may find use in tissue culture applications, as well. Rapid determination of DNA quantity in assays of cell lysates is not readily achievable. FIG. 13 demonstrates the measurement of DNA levels in cells lysates (0.1% NP40 in medium containing 10% FCS) and the linear relationship between DNA concentration and LDH activity detected. Supernatant free DNA quantification was determined (FIG. 13A) as was supernatant LDH activity (FIG. 13B), and the correlation between the two was plotted (FIG. 13C).

Thus, the assays of this invention provide for rapid DNA quantification, and determination of specific activities in cell lysates, providing a more quantitative analysis than has been achievable to date in other rapid assays.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1-43. (canceled)

44. A method of quantifying the nucleic acid concentration in a biological fluid, the method comprising the steps of:
a) mixing a biological fluid sample with a detectable nucleic acid intercalating agent, which agent is a fluorescent agent, wherein said mixing is conducted in the absence of prior nucleic acid extraction;
b) detecting fluorescence emission at a single wavelength of said fluorescent agent; and
c) correlating detection of said agent with a value reflective of the concentration of nucleic acid in said biological fluid sample.

45. The method of claim 44, wherein serially diluted fluid samples are mixed.

46. The method of claim 44, wherein said detecting is conducted with the use of a fluorimeter.

47. The method of claim 44, wherein said detectable nucleic acid intercalating agent comprises SYBR Gold© or SYBR Green©.

48. The method of claim 44, wherein said biological fluid is isolated from a subject and said biological fluid is serum, plasma, a cell lysate or tissue homogenate.

49. The method of claim 44, wherein said method is conducted in parallel to mixing a second biological fluid sample, and said correlating results in said value representing a standard for said method.

50. The method of claim 44, wherein said method further comprises mixing a second fluid sample comprising a known concentration of nucleic acid with said detectable agent and correlating detection with a value equal to said known concentration.

51. The method of claim 50, wherein said correlating includes assigning a value to said biological fluid sample based on the comparative detection with that obtained for said second fluid sample.

52. The method of claim 44, wherein said biological fluid sample comprises urine, blood or a component thereof.

53. The method of claim 44, wherein said biological fluid sample is obtained by lavage of a tissue in a subject.

54. The method of claim 44, wherein said biological fluid is isolated from a subject having or predisposed to a disease or disorder.

55. The method of claim 54, wherein said method further comprises diagnosing the presence of said disease or disorder based on said value obtained.

56. The method of claim 54, wherein said method further comprises predicting the severity of said disease or disorder based on said value obtained.

57. The method of claim 54, wherein said disease or disorder comprises a tissue injury, infection, an inflammatory response, graft or transplant rejection, neoplasia or preneoplasia.

58. A method of quantifying the in vitro nucleic acid concentration in a tissue culture fluid, the method comprising the steps of:
a) mixing a tissue culture fluid sample with a detectable nucleic acid intercalating agent, which agent is a fluorescent agent, wherein said mixing is conducted in the absence of prior nucleic acid extraction;
b) detecting fluorescence emission at a single wavelength of said fluorescent agent; and
c) correlating detection of said moiety with a value reflective of the concentration of nucleic acid in said tissue culture fluid sample.

59. A method of quantifying the residual nucleic acid concentration in a recombinant protein bioreactor fluid, the method comprising the steps of:
a) mixing a fluid sample obtained from a bioreactor for the preparation of recombinant proteins with a detectable nucleic acid intercalating agent, which agent is a fluorescent agent, wherein said mixing is conducted in the absence of prior nucleic acid extraction;
b) detecting fluorescence emission at a single wavelength of said fluorescent agent; and
c) correlating detection of said moiety with a value reflective of the concentration of nucleic acid in said fluid sample.

60. A kit for the quantification of the nucleic acid concentration of a bodily fluid of a subject, said kit comprising:
a) a single detectable nucleic acid intercalating agent, wherein said agent is a fluorescent agent;
b) a diluent; and
c) a series of solutions comprising nucleic acid samples in said diluent, wherein the concentration of each of the nucleic acid samples in said series is known;
whereby a bodily fluid sample is mixed with said detectable nucleic acid intercalating agent in parallel to mixing said agent with said series, and detection of the emission at a single wavelength of said agent in said series serves as a standard for arriving at a value reflective of the concentration of nucleic acid in said bodily fluid sample.

61. The kit of claim 60, optionally comprising a container suitable for accommodating said series of solutions and said bodily fluid sample and wherein said container may by applied to a fluorimeter.

62. The kit of claim 60, wherein said detectable nucleic acid intercalating agent comprises SYBR Gold© or SYBR Green©.

63. The kit of claim 60, wherein said kit comprises a container suitable for the assay of urine, blood or a component thereof, lavage fluid or a combination thereof.
